(19) United States
(12) Patent Application Publication
(10) Pub. No.: US 2025/0257301 A1
(43) Pub. Date: Aug. 14, 2025

(54) MICROFLUIDIC APPARATUS

(71) Applicant: SCREENIN3D LIMITED, Glasgow (GB)

(72) Inventor: Michele ZAGNONI, Glasgow (GB)

(21) Appl. No.: 18/856,409

(22) PCT Filed: Mar. 27, 2023
(86) PCT No.: PCT/GB2023/050778

§ 371 Date: Oct. 11, 2024

(30) Foreign Application Priority Data
Apr. 14, 2022 (GB) 2205532.1

(51) Int. Cl. C12M 3/06; B01L 3/00

(52) U.S. Cl. CPC C12M 23/16; B01L 3/502746; B01L 2200/027; B01L 2300/069; B01L 2300/0816; B01L 2300/0887

(57) ABSTRACT

A microfluidic apparatus for culturing cells comprising: a microfluidic device (10) comprising: a collection reservoir (36) for collecting fluid, wherein the collection reservoir (36) is at least partially closed at a first end and comprises at least one outlet (38) at or adjacent to the at least partially closed first end; a fluid channel and an inlet coupling the fluid channel to the collection reservoir (36); wherein the apparatus further comprises: at least one separable material layer configured to be interfaced with the at least one outlet (38) wherein the at least one separable material layer comprises absorbent properties for controlling fluid flow from the at least one outlet (38).

### FIELD

The present invention relates to a microfluidic device and apparatus, for example, for culturing and screening cells.

### BACKGROUND

Tumours contain a heterogeneous cell population and grow in three dimensions, resulting in the formation of gas, nutrient and drug concentration gradients within tissue. Presently, cancer research and drug testing in industry and academia is mostly conducted on 2D cell monolayers. 2D cell monolayers lack the complexity of real tumours. Furthermore, research is carried out using cell lines, which poorly reflect tumour heterogeneity and genetic diversity. Known techniques, including spinner flasks and hanging drop plates, render screening of limited samples of human tissue (such as biopsies or patient-derived 3D tumoroids) in a medium or high-throughput manner not possible.

WO 2018/189532A1 describes a microfluidic device with an overflow opening.

### SUMMARY

In accordance with a first aspect there is provided a microfluidic apparatus comprising: a microfluidic device comprising: a collection reservoir for collecting fluid, wherein the collection reservoir is at least partially closed at a first end and comprises at least one outlet at or adjacent to the at least partially closed first end; a fluid channel and an inlet coupling the fluid channel to the collection reservoir; wherein the apparatus further comprises: at least one separable material layer configured to be interfaced with the at least one outlet wherein the at least one separable material layer comprises absorbent properties for controlling fluid flow from the at least one outlet. The microfluidic apparatus may be for culturing cells.

The material layer may comprise at least one absorbing and/or non-absorbing portion such that, when the at least one outlet of the device is interfaced with the absorbing and/or non-absorbing portion the material layer has an effect on the fluid flow of the device.

The effect on the fluid flow may comprise at least one of: permitting or preventing fluid flow and/or increasing or at least decreasing fluid flow.

When the at least one outlet is interfaced with an absorbing portion, fluid flow from the collection reservoir to the material layer may be permitted. When interfaced with the non-absorbing portion, fluid flow from the collection reservoir may be prevented or at least decreased. Permitting fluid flow may allow perfusion to be achieved. Preventing fluid flow may allow a static cell culture to be maintained. The separable material layer may comprise wicking properties for wicking fluid from through the at least one outlet.

The collection reservoir may be at least partially closed at the first end by a base portion. The at least one outlet may be for draining fluid from the collection reservoir and wherein at least part of the at least one outlet is provided on or adjacent to the base portion at the first end of the collection reservoir. The base portion may form part of the separable material layer.

The collection reservoir may be at least partially closed at the first end such that the reservoir is configured to collect and at least partially contain the fluid. The collection reservoir may be at least partially closed by the base portion. The at least one outlet may be positioned such that, in use, fluid exits the collection reservoir via the at least one outlet in response to a lateral fluid flow and/or gravity. The inlet may be positioned such that, in use, the collection reservoir is filled with fluid from the at least partially closed end upwards.

The device may further comprises at least one source reservoir. The fluid channel may channel a fluid from the at least one source reservoir to the at least one collection reservoir. The fluid channel may comprise a plurality of chambers or wells for holding cells. The plurality of chambers may be formed underneath and open to the fluid channel. The fluid may comprise compounds and/or proteins.

At least part of the at least one outlet may be aligned with the inlet. At least part of the at least one outlet may be lower than the inlet.

The collection reservoir may comprise at least one side wall extending from the base portion. The inlet may be provided at a first depth on the at least one side wall and at least part of the at least one outlet is provided at substantially the same or a lower depth on the at least one side wall and/or base portion.

The collection reservoir may be formed in a body of the microfluidic device and wherein the at least one outlet is on an outer or external surface of the body.

The at least one outlet may be positioned such that fluid can exit the microfluidic device via the at least one outlet. The body may comprise a lower surface, an upper surface and at least one side surface. At least part of the at least one outlet may be provided in the lower surface and/or the at least one side surface.

The base portion of the collection reservoir may form part of the lower surface. The height of the collection reservoir may be substantially equal to the height of main body. The at least one outlet may comprises a drain at a base of the collection reservoir. The collection reservoir may span at least part of the distance between the lower surface and the upper surface.

The collection reservoir may comprise a depth and wherein: at least part of the at least one outlet is positioned below half of the depth of the collection reservoir; optionally below a quarter of the depth.

The at least one outlet may comprise an aperture provided in the base portion. The aperture may have an area less than half, optionally a quarter, optionally an eighth of the area of the base portion.

The at least one outlet may form part of a void in at least one wall of the collection reservoir, optionally wherein the void spans the depth of the collection reservoir.

The at least one outlet may comprise a cylindrical or a conical shape.

The collection reservoir may comprises a circular shape, as viewed from above or below. The collection reservoir may comprise a notch or semi-circular shape, when viewed form above or below.

The base portion may be of a single piece construction with the collection reservoir.

The microfluidic device may comprise at least a first part and a second, separable base, wherein the separable base is configured to co-operate with the first part to at least partially close the collection reservoir and/or to form the base portion.

At least part of the base portion may form a fluid flow barrier. The at least one outlet may provide a fluidic path for fluid out of the collection reservoir and out of the device. The at least one outlet may be positioned to permit collected fluid in the collection reservoir to exit the body and/or exit the collection reservoir. The base and/or base portion may comprise a base member. The base member may substantially surround the at least one outlet. The at least one outlet may be provided adjacent to the base member. The base member may comprise a semi-circular shape and/or an annular shape.

The device may be configured to be placed on a further surface and the at least one outlet may be positioned at the at least partially closed first end to provide a fluidic path between the collection reservoir and the further surface. The further surface comprises a material layer having absorbent and/or non-absorbent properties.

The apparatus may comprise securing means for securing the material layer with the device, such that, when secured, the at least one outlet is interfaced with the material layer.

The securing means may comprise: at least a base part for receiving the material layer. The securing means may further comprise an upper part for receiving the at least one microfluidic device. The upper part may comprise the at least one microfluidic device.

The securing means may comprise a coupling mechanism for placing the upper part and/or the device into a coupled configuration with the base part, such that, when in the coupled configuration, the material layer received in the base part is interfaced with at least one at least one outlet of the at least one microfluidic device.

The coupling mechanism may comprise mechanical or magnetic means.

The securing means may further comprises one or more alignment features. The alignment features may comprise, for example, alignment pins and/or visual markers. The securing means may further comprise one or more mating features.

At least part of the securing means may comprise at least one dimension corresponding to a known well-plate format.

The upper part and/or base part may comprise dimensions of a known well plate format, for example, a 96 or 384 well plate format. The upper part may be configured to hold a plurality of microfluidic devices.

The device may comprise at least one dimension selected to correspond to a known well plate format. The device may comprise such a source reservoir, and the distance between the source reservoir and the collection reservoir may correspond to a pre-determined distance of a well plate format.

The at least one outlet may comprise a plurality of outlets. The plurality of outlets May comprise at least one pair of outlets arranged at opposing sides of the collection reservoir. At least one, optionally all, of the plurality of outlets may be provided at and/or intersecting with a side wall of the collection reservoir.

The at least one outlet may be provided in an annular region defined in the base portion between half the radius of the base portion and the side wall, optionally between three quarters of the radius of the base portion to the side wall, further optionally, substantially at the side wall.

The at least one outlet may be provided in an annular region defined in the base portion beyond half the radius of the base portion, optionally beyond three quarters of the radius of the base portion wall, further optionally, substantially at the side wall. The at least one outlet may be provided in an annular region having a width substantially equal to the width of the outlet. The at least one outlet may be provided in an annular region having a width substantially equal to the width of the outlet and provided at an outer edge of the base portion and/or contiguous to the side wall.

In accordance with a second aspect that may be provided independently, there is provided a microfluidic device comprising: a collection reservoir for collecting fluid, wherein the collection reservoir is at least partially closed at a first end; a fluid channel and an inlet coupling the fluid channel to the collection reservoir; wherein the collection reservoir further comprises an at least one outlet for draining fluid from the collection reservoir, wherein at least part of the at least one outlet is provided at on or adjacent to the first end of the collection reservoir. The collection reservoir may be at least partially closed by a base portion. The at least one outlet may be provided on or through or adjacent to the base portion.

In accordance with a third aspect, there is provided, a material layer for use with at least one microfluidic device, wherein the material layer comprises a plurality of absorbing and/or non-absorbing portions distributed to be interfaced with a corresponding plurality of outlets of one or more microfluidic devices. The at least one microfluidic device may be provided in accordance with the second aspect.

In accordance with a fourth aspect, that may be provided independently, there is provided a method of use of a microfluidic device, the microfluidic device comprising a collection reservoir for collecting fluid, wherein the collection reservoir is at least partially closed at a first end and comprises at least one outlet at or adjacent to the at least partially closed first end and a fluid channel and an inlet coupling the fluid channel to the collection reservoir; wherein the method comprises at least one of: a) providing the microfluidic device on a first material layer, wherein the first material layer comprises a non-absorbing portion such that at least one outlet of the microfluidic device is interfaced with the non-absorbing portion thereby to prevent or at least reduce flow from the microfluidic device; b) providing the microfluidic device on a second material layer, wherein the second separate material layer comprises an absorbing portion such that the at least one outlet at the base of the microfluidic device is interfaced with the non-absorbing portion thereby to permit or at least increase fluid flow from the microfluidic device.

In accordance with a fifth aspect, that may be provided independently, there is provided, a kit of parts comprising: one or more microfluidic devices according to the second aspect and a separable material layer according to the third aspect.

Features in one aspect may be provided as features in any other aspect as appropriate. For example, features of the first aspect may be provided as features of the second aspect. For example, features of the device or apparatus may be provided as features of a method and vice versa. Any feature or features in one aspect may be provided in combination with any suitable feature or features in any other aspect.

### BRIEF DESCRIPTION OF THE FIGURES

Various aspects of the invention will now be described by way of example only, and with reference to the accompanying drawings, of which:

FIG. 1 is a perspective view a microfluidic device in accordance with embodiments;

FIG. 2 illustrates the collection reservoir of the microfluidic device of FIG. 1;

FIG. 3 illustrates an outlet of the microfluidic device of FIG. 1;

FIG. 4 illustrates an outlet of the microfluidic device in accordance with a further embodiment;

FIG. 5 depicts the microfluidic device with one or more layers having absorbing and non-absorbing properties;

FIG. 6 depicts a microfluidic apparatus, in accordance with an embodiment;

FIG. 7 depicts a microfluidic apparatus, in accordance with a further embodiment;

FIG. 8 depicts a microfluidic device and further layer, in accordance with a further embodiment;

FIG. 9 depicts a plurality of microfluidic devices and a further layer, in accordance with a further embodiment;

FIG. 10 depicts part of a microfluidic apparatus in accordance with a further embodiment;

FIG. 11 depicts a microfluidic device in accordance with a further embodiment, and

FIG. 12 depicts a microfluidic device in accordance with a further embodiment.

### DETAILED DESCRIPTION

A microfluidic device and apparatus is described herein. The device is suitable for culturing and/or screening cells. As described in the following, the device has an outlet and is configured to be used with one or more further materials to control the flow from the outlet to allow the establishment and maintenance of a cell culture in the device. For example, by interfacing the outlet with an absorbing portion of a further layer, fluid flow is permitting in the device, which may allow perfusion and/or drug concentration gradients (and combination of such gradients) to be achieved. As a further example, by interfacing the outlet with a non-absorbing portion of a further layer, a static cell culture may be maintained. The apparatus may include, in some embodiments, may allow fluid flow of more than one device to be controlled simultaneously by using the same material layer.

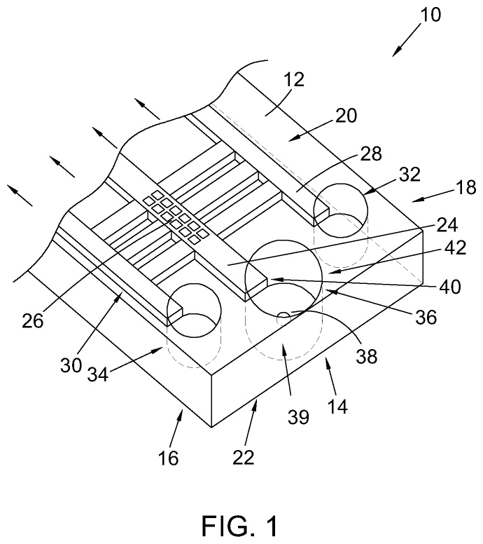

FIG. 1 shows a microfluidic device 10. In the present device, the device 10 has a body 12 having a prism or cartridge shape, having three pairs of parallel, external facing surfaces of the body. The device 10 is rectangular, when viewed from above or below. The surfaces include two, parallel end surfaces referred to as a proximal end surface 14 and a distal end surface (not shown) and two lateral side surfaces: a first lateral surface 16 and a second lateral surface 18. The device 10 also has a rectangular upper surface 20 and a rectangular lower surface 22. The device 10 has a length, in a longitudinal direction that corresponds to the distance between the parallel end surfaces. The device 10 also has a width, in a lateral direction, that corresponds to the distance between the first lateral surface 16 and the second lateral surface 18. The device 10 also has a height that corresponds to the distance between the upper surface 20 and lower surface 22.

In the present embodiment, the device 10 has longitudinal and lateral symmetry. The device 10 has a first line of symmetry about the mid-point of the lateral direction and a second line of symmetry about the mid-point of the longitudinal direction. As such, only a first part including the proximal end surface of the device 10 is depicted in FIG. 1. However, it will be understood that the following description applies to corresponding elements of the distance part of the device 10.

The device 10 has a central fluid channel 24 extending along part of its length, in the longitudinal direction. An array of chambers, in this embodiment micro-wells 26, suitable, for example, for the culture of 3D multicellular spheroids, are provided in a bed of the central fluid channel 24. In the present embodiment, the array of micro-wells 26 are formed such that the micro-wells are provided underneath the bed of the central channel 24 and open at an upper end to the central channel 24.

Extending substantially along, and parallel to, the first and second lateral surfaces of the device 10 and provided on opposing, lateral sides of the central channel 24 there are a first lateral channel 28 and a second lateral channel 30. The first lateral channel 28 and second lateral channel 30 have equal dimensions in the longitudinal direction (length), lateral direction (width) and an equal depth or height. The first lateral channel 28 extends parallel to the second lateral channel 30 and parallel to the central channel 24. The first and second lateral channels extend in the longitudinal direction and can be considered to have a proximal and distal end. Likewise, the central channel 26 extends in a longitudinal direction and can be considered to have a proximal and a distal end. The terms proximal and distal ends refer to the ends of the channel nearest to the proximal end surface 14 and the distal end surface (not shown).

Each channel 24, 28 and 30 is provided within the body 12 of the device 10. Relative to the upper surface 20 of the device 10, the lateral channels 28 and 30 are provided at a side channel depth. Likewise, the central channel 24 is provided at a central channel depth.

A number of reservoirs are provided in the body 12 of the device 10 as follows. Each channel 24, 28, 30 has two associated cylindrical reservoirs provided at either end of the channel. First lateral channel 28 is coupled to a first associated source reservoir 32 at its proximal end at a second associated source reservoir (not shown) at its distal end. Likewise, second lateral channel 30 is coupled to a first associated source reservoir 34 at its proximal end and a second associated source reservoir at its distal end (not shown). Likewise, central channel 26 is coupled to a first associated collection reservoir 36 at its proximal end and a second associated collection reservoir at its distal end (not shown).

While FIG. 1 depicts two source reservoirs (first source reservoir 32 and second source reservoir 34) and a collection reservoir 36, however, it will be understood, that due to the symmetry of the device 10, the device 10 has two further source reservoirs and a further collection reservoir not shown in FIG. 1. The collection reservoir(s) may also be referred to as central reservoirs.

The reservoirs are coupled to their associated channel such that the reservoirs can collect fluid from and/or provide fluid for their associated channels. The reservoirs can also store fluid. When fluid is in a channel and stored in the associated reservoirs, the reservoirs at both ends of the channel may act to exert a hydrostatic pressure on fluid in the channel at both ends, thereby holding the fluid in the channel at a hydrostatic pressure.

The reservoirs are wells formed in the body of the device and each reservoir may therefore be referred to as a well. In the present embodiment, each reservoir is cylindrical and extends from the upper surface 20 of the device 10 to the respective depth of their associated channels. The reservoirs are open at the upper surface 20 of the device 10, such that the openings of the reservoirs provide an inlet to their associated channel. The volume and therefore capacity for holding and storing fluid in the reservoir may be determined by reservoir depth and surface area of the reservoir. The central channel 24 and/or the lateral channels may be referred to as fluid supply channels. The central channel 24 has a first central reservoir 36 at a first end (the proximal end) and a second central reservoir (not shown) at its second end. Both central reservoirs have an equal volume and therefore equal capacity for holding fluid.

In the following a description of an outlet for the central channel 24 is provided. It will be understood that, a corresponding outlet is provide in the further central reservoir (not shown in FIG. 1).

In the present embodiment, the first collection reservoir 36 has an outlet 38 provided at a lower end of the reservoir. In further detail, in the present embodiment, the collection reservoir 36 is partially closed at a first end by a base portion 39. The outlet 38 is provided on the base portion 39 in the form of an aperture formed through the base portion 39. The collection reservoir 36 is therefore partially closed by the base portion 39. The central reservoir is also defined by a cylindrical side wall 42. The central reservoir 36 may be considered as a hollow cylinder formed through body 12 that is partially closed at a first end (the end of the lower surface 22) and open at a second end (the end at the upper surface 20). The central reservoir has an inlet 40 at the proximal end of the central channel 24. The inlet 40 couples the central channel 24 to the first central reservoir 36. It will be understood that, although not shown in the FIG. 1, the second collection reservoir at the distal end of the device also has an inlet, at the distal end of the central channel 24 and has an outlet corresponding to the outlet 38 of reservoir 36.

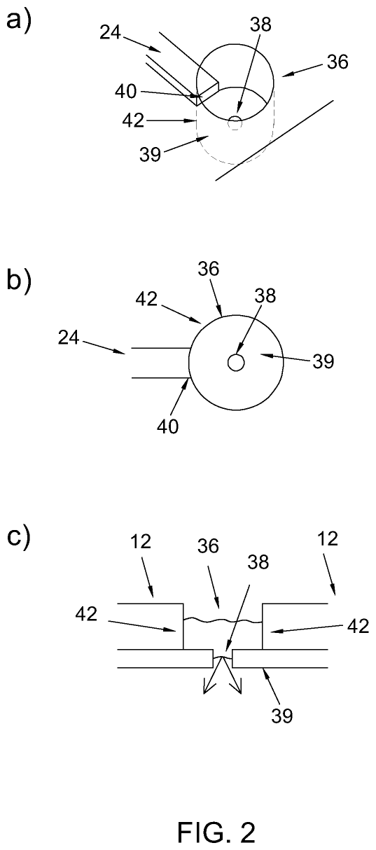

FIG. 2 provides a detailed view of the outlet of the central reservoir. FIG. 2(a) shows a perspective view of the central reservoir; FIG. 2(b) shows an elevated view of the central reservoir (from above) and FIG. 2(c) shows a cross-section, side view of the central reservoir. FIG. 2 depicts the central reservoir 36, the side wall 42, the base portion 39 and the inlet 40 coupling the central channel 24 to the central reservoir 36.

In this embodiment, as described above, the central reservoir 36 has a cylindrical shape. The central reservoir 36 is open at its upper end. The reservoir has a uniform width such that the base portion 39 has a diameter equal to the diameter of the open upper end and the inlet 40 is provided where it intersects with the central channel 24. The side wall 42 of the central reservoir extend upwards from the base portion 39. At an upper end of the collection reservoir 38 is a further opening that provides an inlet and has a size corresponding to the cross-sectional area (when viewed from above) of the collection reservoir. In use, this opening may be covered. It will be understood that, in use, the desired level of fluid in the collection reservoir is below the height of the further opening.

As depicted in FIG. 2(b), the base portion 39, in the present embodiment is formed of a solid base element having an annular shape that surrounds the outlet 38. As is seen in FIG. 2, the outlet 38 at provided at the centre of the base portion 39. The outlet is provided on an external surface (the lower surface) of the microfluidic body and provides access between an internal part of the microfluidic device and the external environment.

FIG. 2(c) is a side view of the collection reservoir 36. FIG. 2(c) depicts collection reservoir as a cylindrical hollow formed in the body 12 of the device. At the lower end, the base portion 39 and outlet 38 through the base portion 39 is depicted. As depicted in FIG. 2(c), in the present embodiment, the base portion 39 forms part of a base of the body.

As shown in FIG. 2(c), in the absence of any other material or surface below the outlet, fluid flows freely from the collection reservoir via the outlet 38. As can be seen in FIG. 2(c), the collection reservoir is partially closed at the first end by the base portion and the outlet allows the fluid to flow from the device using a combination of, for example, lateral fluid flow and/or gravity. The inlet is positioned at the base portion so that the collection reservoir is filled by fluid from the fluid channel from the base portion upwards.

It will be understood that, in other embodiments, the base portion may be formed as part of a separable base. In addition, it will be understood that the outlet may not be provided at a centre but may be provided at alternative position. In addition, while only a single outlet is depicted, it will be understood that more than one outlet may be provided at the base of the reservoir. In some embodiments, the aperture has a size selected relative to the size of the base portion/base of the reservoir. Suitable values may include an area that is half that of the collection reservoir base. In some embodiments, the area is a quarter or an eight of the area of the base.

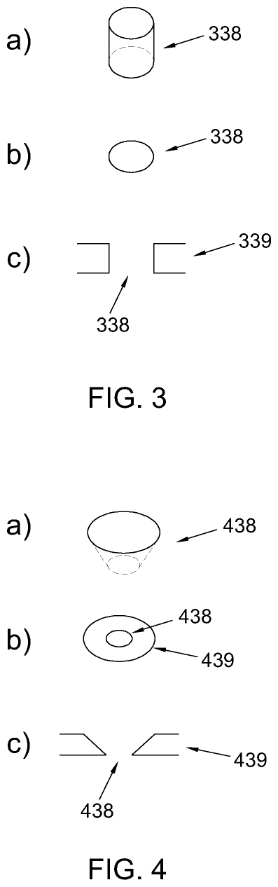

The outlet 38 described above has a circular shape. It will be understood that the outlet may have a different shape in different embodiments. FIG. 3 depicts an outlet 338 having a first, cylindrical shape in a base portion 339. FIG. 4 depicts a second outlet 438 having a bevelled or conical shape in a base portion 439.

FIGS. 3(a), (b) and (c) depicts a perspective, above-view and side view of an outlet, in accordance with embodiments. FIGS. 4(a), (b) and (c) depict a perspective, above-view and side view of an alternative outlet, accordance with embodiments.

As shown in FIG. 3, the outlet 338 has a circular cross-section. As the outlet is formed through the base portion 339, the outlet 338 has a depth equal to the depth of the base portion. The outlet 338 can be considered as a cylindrical aperture through the base portion 339, having a constant cross section through the base portion. The outlet 338 may have a diameter in the range 1 to 2 mm. In the present embodiment, the outlet 338 has a diameter of 1 mm.

FIG. 4 depicts a conical shaped outlet 438 in accordance with an embodiment. FIG. 4(a) is a perspective view of the outlet. FIG. 4(b) is an elevated view of the outlet and FIG. 4(c) is a side view of the outlet. While FIG. 4 depicts a conical shaped outlet, it will be understood that other shapes may be formed through the base layer to provide fluid drainage. Outlet 438 has a conical shape (as depicted in FIG. 4(a)). FIG. 4(b) and FIG. 4(c) depict the base portion 439 having bevelled edges to form the outlet 438.

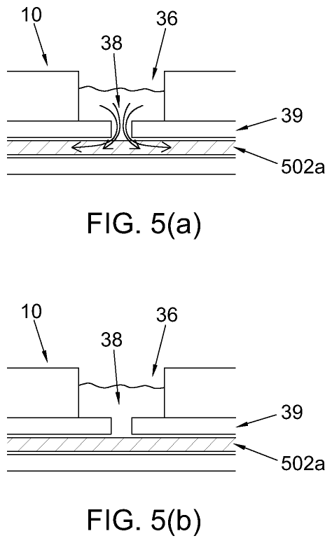

As described in the following, the microfluidic device is for use with a further material layer, the material layer having absorbing and non-absorbing properties that control the flow of fluid from the device. For the purposes of illustration, FIG. 5(a) depicts the microfluidic device together with a first separable, absorbing layer 502a and FIG. 5(b) depicts the microfluidic device with a second non-absorbing material layer.

In this embodiment, the device 10 is placed on either the absorbing material layer or the non-absorbing layer such that the outlet 38 interfaces with the absorbing/non-absorbing material of the material layer. In this embodiment, the base portion 39 lies on and in contact an upper surface of the material layer.

In FIG. 5(a) the absorbing layer 502a has absorbent properties that act to permit flow from the collection reservoir via the outlet 38. In this configuration, the outlet 38 is interfaced with an absorbing portion of the absorbing layer 502a. Fluid collected in the collection reservoir thus flows through the outlet 38 to the absorbing layer 502a. In this configuration, the outlet 38 provides a drain from the device to the external environment.

In FIG. 5(b) the non-absorbing layer 502b has non-absorbent properties that act to prevent flow from the collection reservoir 36 via the outlet 38. In this configuration, fluid from the collection reservoir 36 is prevented from draining to the material layer and is retained in the collection reservoir 36. In such a configuration, the material layer and the base portion form a barrier at the base of the collection reservoir. In the present embodiment, the non-absorbing layer and base portion form an impermeable barrier thus closing the collection reservoir at its base end.

By providing a separable absorbing/non-absorbing layer together with the device, fluid flow can be controlled. While the above-described embodiments described permitting and preventing fluid flow, it will be understood that the material layer can have properties such that fluid flow is increased or decreased. For example, the fluid flow rates may be controlled by the absorbing properties of the material layer.

In addition, while interchangeable layers are described it will be understood that, in some embodiment, a single material layer having absorbing/non-absorbing portions may be provided. It will also be understood that by interfacing the outlet with absorbing and non-absorbing materials, the collection reservoir may be effectively opened and closed.

As a non-limiting example, in use, the device is provided on the absorbing layer 502a to permit fluid flow through the device. By permitting fluid flow, a compound gradient, or multiple compound gradients combined, can be established across the plurality of micro-wells. After a set amount of time, the absorbing layer 502a is then swapped to the non-absorbing layer 502b and the device is provided on the non-absorbing layer. The non-absorbing layer prevents fluid flow thus allowing the established compound concentration gradient to be substantially maintained over a desired period of time. As a further example, by prevent fluid flow, the non-absorbing layer may prevent flow thereby to allow for a cell culture process to be performed. The material layer may be any suitably porous/non-porous material. The material layer may comprise, for example, paper and/or cotton and/or textiles and/or fibre. The degree of absorption of the material layer is dependent on material properties of the layer, for example, the porosity of the material.

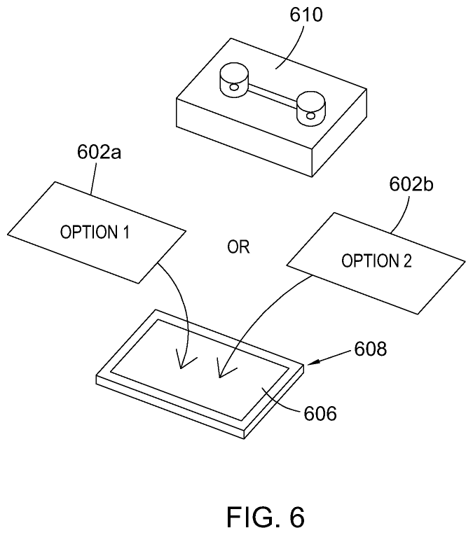

FIG. 6 depicts a structure for holding a microfluidic device 610 in accordance with an embodiment. Microfluidic device 610 can be considered to have common features with device 10. FIG. 6 is an exploded view of an apparatus having a microfluidic device 610, a separate removable material layer either absorbing layer 602a or non-absorbing layer 602b, and a base unit 606. The structure of FIG. 6 can be considered as an example of a securing means for securing the microfluidic device in place with the separable material layer. As depicted in FIG. 6, the base unit 606 is sized to receive the removable material layer. Base unit 606 has a rim feature that retains the material layer in place. The void defined by rim feature 608 has the same dimensions as both material layers and the device 610.

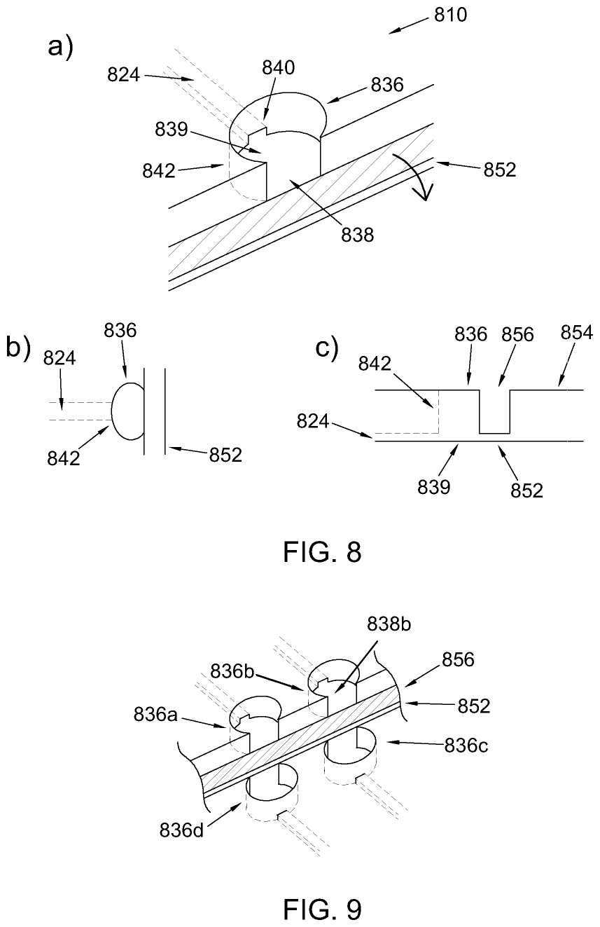

It will be understood, that on assembly, the material layer is received in the void of the base unit 606 and the device 610 provided on top of the material layer. When the device 610 is placed on the material layer, the outlet of the device 10 is therefore interfaced with an absorbing/non-absorbing portion of the material layer, substantially as described with reference to FIG. 5 and, for example, FIGS. 8 and 9.

Device 610 depicts a device with two wells (correspond to, for example, the two central reservoirs described above). In some embodiments, at least one dimension of the device is selected so that the device 610 corresponds to a known well plate format (for example, 96 or 384 well). As a non-limiting example, the distance between the respective centres of the central reservoirs is selected to correspond to a known well plate form (for example, 9 mm or 4.5 mm). Part of the securing means (for example, the base unit) may have dimensions that also correspond to known well plate formats. For example, the overall dimensions may correspond to a known standard size (for example, 85.48×127.76 mm). By selecting a device having such dimensions, the device or a plurality of devices arranged together may be usable with known sample processing apparatuses.

In some embodiments, one or more alignment features are provided. These may include visual markers on the first and second parts that allow a user to visually inspect when the two parts are correctly aligned.

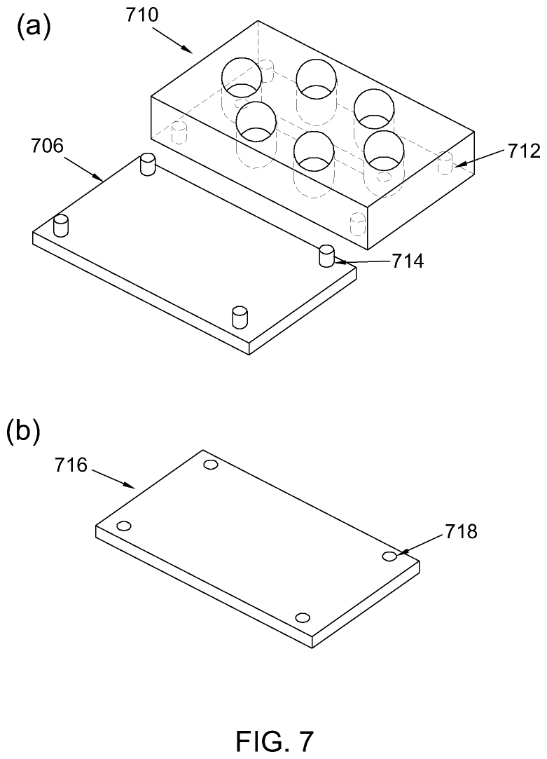

FIG. 7(a) depicts an apparatus with mating features, in accordance with an embodiment. FIG. 7(b) depicts an apparatus with mating features, in accordance with a further embodiment. The mating features may provide allow a user to visually align and also to provide a physical coupling of parts of the apparatus together.

FIG. 7 depicts a structure for holding a microfluidic device 710 in accordance with an embodiment. FIG. 7 is an exploded view of an apparatus having a microfluidic device 710 and a base part (or base unit) 706. The device 710 and base part 706 correspond to the device and base part as described with reference to FIG. 6.

Base part 706 and device 710 have mating features for mating the base part 706 with the device 710. In further detail, the base part 706 has a plurality of mating features configured to mate with a corresponding plurality of complementary mating features on the device 710. The base part 706 is configured to receive a separable material layer and, while the separable material layer is not depicted in FIG. 7, it will be understood that the mating features of FIG. 7 provide a securing means for securing the separable material layer between the base part and the device. The structure of FIG. 7 can therefore be considered as an example of a securing means for securing the microfluidic device in place with a separable material layer.

In this embodiment, the mating features of the base part 706 are in the form of protrusions (a first protrusion 714 is depicted in FIG. 7) formed in the upper surface of the base part 706. In this embodiment, the complementary mating features of the device 710 are notches (a first notch 712 is depicted in FIG. 7). The notches are sized and shaped to complement the protrusions. In use, first notch 712 couples with first protrusion 714 by receiving the first protrusion 714. The notches are formed in the lower part of the device 710. In the present embodiment, four protrusions are provided, each protrusion in one of the four corner regions of the upper part 704. Four corresponding notches are provided in the corresponding corner regions of the device 710.

It will be understood that other distributions and numbers of mating features may be suitable, in other embodiments. While the protrusions depicted in FIG. 7 are cylindrical in shape and the notches have corresponding cylindrical shape, it will be understood that the protrusions and notches can be any suitable complementary shape that secure the upper part to the device. The mating features offer mechanical coupling between the upper part and the device. In use, the mating features prevent movement of the device with respect to the upper part. The mating feature may also provide additional alignment to facilitate correct alignment of the upper part to the device.

FIG. 7(b) depicts a base part 716 in accordance with another embodiment. In FIG. 7(b) the base part 716 has magnetic mating features configured to magnetically couple the base part 716 and the device (not shown). A first magnetic feature 718 is depicted in FIG. 7(b). It will be understood that, in this embodiment, a corresponding magnetic feature is provided in the device (not shown). While only part of the base part is described as magnetic, in further embodiments, the entire base part and/or device may have magnetic properties. In some embodiments, mating features may provide both mechanical and magnetic coupling (for example, magnetic protrusion and notch pairs).

FIG. 8 depicts an outlet 838 of a microfluidic device 810 in accordance with an alternative embodiment. It will be understood that, aside from the features described in the following, microfluidic device 810 is substantially the same as device 10 and features in common between device 810 and device 10 are not described in detail here. In particular, microfluidic device 810 has a central channel 824 (corresponding to central channel 24) that is coupled to collection reservoir 836 by inlet 840.

The collection reservoir is partially defined by side wall 842. However, in contrast to device 10, the side wall 842 does not completely surrounds the base portion 839 on all sides but only partially surrounds the base portion 839 and a void is formed. A void is formed in part of the side wall 842. The base portion 839 has a semi-circular-like shape. The void spans the depth of the body of device, corresponding to the height of the collection reservoir 836. The void therefore provides an outlet 838 for the collection reservoir. In contrast to device 10, the outlet is not formed in the base portion 839, but instead the lower part of the outlet 838 is adjacent to the base portion 839. The outlet forms part of a void in the side wall of the collection reservoir. The outlet 838 is such that, in use, fluid flows in a lateral or horizontal direction out of the collection reservoir.

As shown in FIG. 8(b) the outlet 838 is at an opposing side of the collection reservoir to the outlet. While the outlet 838 spans the depth of the device, a lower part of the outlet is aligned, in height, with the depth of the channel 824 and inlet 840. The outlet is provided on an external surface (the proximal surface) of the microfluidic body and provides access between an internal part of the microfluidic device and the external environment.

FIG. 8(c) depicts a side view of the collection reservoir 836. The channel 824 is coupled to the collection reservoir 836. As can be seen from FIG. 8(a) to (c), the collection reservoir is open at an end opposing the inlet, such that fluid collected in the collection reservoir may flow from the collection reservoir.

FIG. 8(a) depicts an absorbing portion in the form of an absorbing strip 852 of a further material layer on which the device 810 is provided. The outer wall of the device 810 and the further wall 854 form a fluid channel. In use, when interfaced with the absorbing strip 852, fluid in the collection reservoir flows in a first, longitudinal direction out of outlet 830 to the absorbing strip 852. In the configuration with the absorbing layer, the fluid is drawn (via a lateral flow) from the collection reservoir via the outlet.

FIG. 8(c) depicts a further boundary for the fluid flow in the form of a further wall 854. This wall may a wall of, for example, a base unit for holding the device or a further device (for example, see FIG. 9). The further wall 854 and the proximal surface of the device 810 form an external fluid flow channel 856. The external fluid flow channel 856 is external to the device. At the bottom of the external fluid flow channel 856 is the fluid absorbing strip 852 (or non-absorbing portion of a further material layer). The further wall 854 provides a boundary to fluid flowing from the outlet such that fluid flowing moves along the fluid flow channel.

FIG. 9 depicts a configuration in which more than one outlet is interfaced with the single absorbing strip 852. In FIG. 9, the external fluid flow channel 856 is formed by a plurality of devices. FIG. 9 depicts four collection reservoirs (836a, 836b, 836c and 836d) arranged so that each of their corresponding outlets is interfaced with an absorbing strip 852 of a further material layer. Only outlet 838b of reservoir 836b is labelled in FIG. 9 for clarity. The outlets are arranged on either side of the absorbing strip such that, in use, when interfaced with the absorbing strip, fluid flows from each of the collection reservoirs out of their respective reservoirs via the absorbing strip.

The above description of specific embodiments is made by way of example only and not for the purposes of limitation.

As a non-limiting example, in some embodiments, the microfluidic device 10 is a multi-layered structure and may be formed from two or more co-operating parts including, for example, a base. The base may comprise the base portion. Alternatively, the device may be a monolithic structure. In some embodiments, at least the collection reservoir is a single piece construction with the base portion.

The assemblies depicted in, for example, FIG. 6 and FIG. 7 provides examples of a securing means and alignment features for securing the material layer with the unit between two parts of the assembly, for example, a base part and the device itself. In further embodiments, a corresponding upper part is provided for holding one or more microfluidic devices. In some embodiments, securing means is provided for holding a plurality of microfluidic devices together with a single material layer so that the outlets of the devices are interfaced with the same layer. In such embodiments, the material layer is sandwiched between the upper part and the base part.

An example of such an apparatus has an upper part for holding the devices, a base that holds the material and a coupling mechanism for coupling the upper part to the base part. When in the coupled configuration, the outlets of the devices are interfaced with the material layer. The coupling mechanism may include suitable mechanical or magnetic means. For example, a mating arrangement for mechanical and/or magnetic coupling of the two parts. Such an apparatus allows a single layer to be used with more than one device at the same time. As such, fluid flow may be controlled simultaneously for each device by simply swapping a single layer of material.

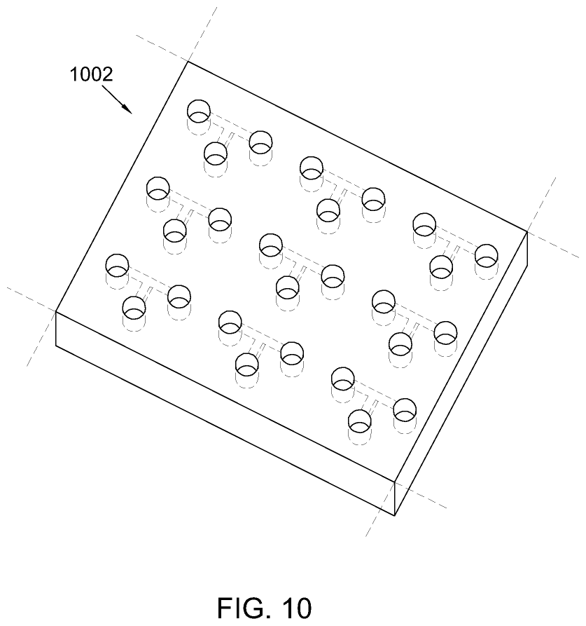

FIG. 10 depicts an apparatus in which an upper part holds more than one device, in accordance with an embodiment. FIG. 10 depicts an upper part 1002 holding a plurality of microfluidic devices. In this embodiment, the upper part 1002 has a structure for holding the devices and the upper part is configured to be secured to a corresponding base part (for example, using one or more mating features or other suitable securing mechanism) thereby to interface the outlets with the material layer. In these embodiments, the plurality of outlets of an array of microfluidic devices is interfaced with the material layer.

In some embodiments, the upper part is not holding more than one device but can be considered as a single assembled structure that provides the function of more than one device. For example, a single device that has a plurality of outlets, reservoirs and corresponding channels that is interfaced with a single material layer.

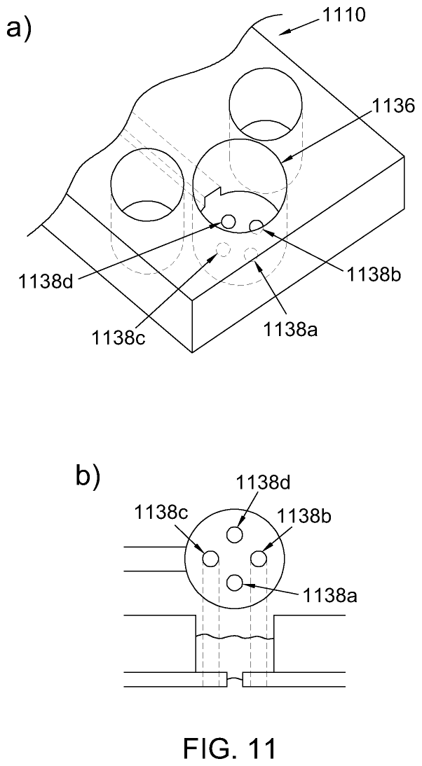

In further embodiment, more than one outlet is provided on the base portion. FIG. 11(a) shows a device 1110 in accordance with a further embodiment. For clarity, FIG. 11(a) depicts a first outlet 1138a, a second outlet 1138b, a third outlet 1138c and a fourth outlet 1138d provided in a base portion 1139 of the collection reservoir 1139. FIG. 11(b) depicts the device 1110 and outlets from above and the side.

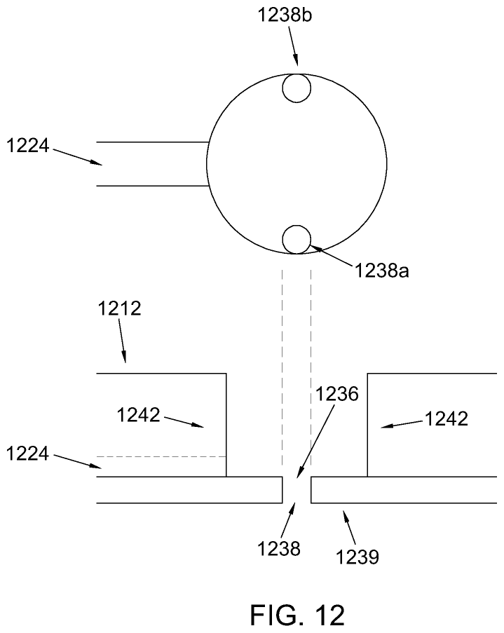

While FIG. 11 depicts a device with four outlets on the base portion, it will be understood that alternative configurations of outlets may be provided in accordance with further embodiments. FIG. 12 shows part of a device in accordance with a further embodiment. The upper figure of FIG. 12 depicts part of the device, as viewed from a position above the device. The lower figure of FIG. 12 depicts the part of the device as viewed from a position at a side of the device. The device has the following elements that are provided substantially as described with reference to, for example, FIGS. 1 and 2: a body 1212, a central fluid channel 1224, a side wall 1242, a collection reservoir 1236 and a base portion 1239. In place of the single outlet of FIG. 1 and the four outlets of FIG. 11, FIG. 12 depicts a first outlet 1238a and a second outlet 1238b. The pair of outlets are provided at opposing sides of the base portion 1239. The pair of outlets are provided at the edges of the base portion 1239. The two outlets form distinct openings in the base portion and are separate from the side wall. However, it will be understood that in further embodiments, one or more of the plurality of outlets may overlap with the sidewall, when viewed from above and form an opening through the side wall.

It will be understood that FIGS. 11 and 12 are examples of configurations of one or more outlets on the base portion 1239. The outlets may be provided substantially at an edge of the base portion 1239, for example, in an annular region defined at the edge of the base portion 1239. Such an annular region may have a width substantially equal to the width of the outlet and be provided at an outer edge of the base portion and/or contiguous to the side wall. The at least one outlet may be provided in an annular region defined in the base portion beyond half the radius of the base portion, optionally beyond three quarters of the radius of the base portion wall, further optionally, substantially at the side wall. For example, the outlets may be provided in an annular region that is defined between half of the radius of the base portion and the side wall or three quarters of the radius of the base portion and the side wall. In some embodiments, the outlets may be provided contiguous to or in contact with the side wall.

While FIG. 1 depicts a single outlet, FIG. 12 depicts two outlets and FIG. 11 depicts four outlets, it will be understood that other configurations and numbers of outlets are possible in further embodiments. For example, more than one pair of outlets may be provided where each outlet of the pair is provided at an opposing side of the collection reservoir. In further non-limiting examples, at least one, optionally all, of the plurality of outlets are provided at or proximal to a side wall of the collection reservoir. In a further example, some or all of the outlets form an opening through the base only. In a further example, some or all of the outlets intersect with the base and the side wall to form an opening through the base and the side wall.

In the above-described embodiments, interfacing of material and outlets is described. It will be understood that, in some embodiments, the material layer is sufficiently close to the outlet that the material layer has an effect on the fluid flow from the outlet.

Furthermore, while the above-described devices can be used, for example, for cell culturing/growth or screening purposes, the device may have other uses. For example, the device may be used for supernatant analysis.

In the above-described embodiments, one or more outlets in a base portion are described. In some embodiments, an outlet is provided at the first end that has a size corresponding to the size of the collection reservoir. In such embodiments, the collection reservoir may be closed at the first end by the separable material layer.

As described above, the material layer may be any suitably porous/non-porous material. The material layer may comprise, for example, paper and/or cotton and/or textiles and/or fibre. The degree of absorption of the material layer is dependent on at least the material properties of the layer, for example, the porosity of the material. In further embodiments, the degree of absorption of the material layer may be varied by using material layers (or portions of material layers) having different grades of porosity thus to control the fluid absorbing rate. The fluid absorbing rate has an effect on fluid flow for similar hydrostatic pressure values in the reservoirs. In some embodiments, the material has hydrophobic properties, for example, to prevent fluid flow. The thickness of the material layer may also determine how much fluid can be absorbed.

In the above-described embodiments, a device with two collection reservoirs is depicted (for example, in FIG. 1). However, it will be understood that devices with different numbers and/or configurations of collection reservoirs and fluid supply channels can be provided in accordance with embodiments.

It will be clear to the skilled person that modifications of detail may be made within the scope of the invention.

What is claimed is:

1.-26. (canceled)

27. A microfluidic apparatus for culturing cells comprising:
a microfluidic device comprising:
a collection reservoir for collecting fluid, wherein the collection reservoir is at least partially closed at a first end and comprises at least one outlet at or adjacent to the at least partially closed first end;
a fluid channel and an inlet coupling the fluid channel to the collection reservoir; wherein the apparatus further comprises:
at least one separable material layer configured to be interfaced with the at least one outlet wherein the at least one separable material layer comprises absorbent properties for controlling fluid flow from the at least one outlet.

28. The apparatus as claimed in claim 27, wherein the material layer comprises at least one absorbing and/or non-absorbing portion such that, when the at least one outlet of the device is interfaced with the absorbing and/or non-absorbing portion the material layer has an effect on the fluid flow of the device.

29. The apparatus as claimed in claim 27, wherein the effect on the fluid flow comprises at least one of: permitting or preventing fluid flow and/or increasing or at least decreasing fluid flow.

30. The apparatus as claimed in claim 27, wherein the collection reservoir is at least partially closed at the first end by a base portion;
wherein the at least one outlet is for draining fluid from the collection reservoir and wherein at least part of the at least one outlet is provided on or adjacent to the base portion at the first end of the collection reservoir.

31. The apparatus as claimed in claim 27, wherein at least part of the at least one outlet is aligned with or lower than the inlet.

32. The apparatus as claimed in claim 27, wherein the collection reservoir comprises at least one side wall extending from the base portion, and wherein the inlet is provided at a first depth on the at least one side wall and at least part of the at least one outlet is provided at substantially the same or a lower depth on the at least one side wall and/or base portion.

33. The apparatus as claimed in claim 27, wherein the collection reservoir is formed in a body of the microfluidic device and wherein the at least one outlet is on an outer or external surface of the body.

34. The apparatus as claimed in claim 30, wherein the at least one outlet comprises an aperture provided in the base portion.

35. The apparatus as claimed in claim 34, wherein the aperture has an area less than at least one of a), b), c):
a) half;
b) a quarter;
c) an eighth of the area of the base portion.

36. The apparatus as claimed in claim 27, wherein at least one of a), b), c):
a) the at least one outlet forms part of a void in at least one wall of the collection reservoir and wherein the void spans the depth of the collection reservoir;
b), wherein the at least one outlet comprises a cylindrical or a conical shape.

37. The apparatus as claimed in claim 30, wherein at least one of a), b), c):
a) the base portion is of a single piece construction with the collection reservoir;
b) the microfluidic device comprises at least a first part and a second, separable base, wherein the separable base part is configured to co-operate with the first part to at least partially close the collection reservoir and/or to form the base portion.

38. The apparatus according to claim 27, further comprising securing means for securing the material layer with the device, such that, when secured, the at least one outlet is interfaced with the material layer.

39. The apparatus according to claim 38, wherein the securing means comprises:
at least a base part for receiving the material layer.
and further comprises:
a coupling mechanism for placing at least one of the device and an upper part holding the device into a coupled configuration with the base part, such that, when in the coupled configuration, the material layer received in the base part is interfaced with the at least one outlet.

40. The apparatus as claimed in claim 39, wherein at least one of a), b):
a) the coupling mechanism comprises mechanical or magnetic means;
b) wherein the securing means further comprises one or more alignment features comprising at least one of a), b): a) alignment pins; b) visual markers.

41. The apparatus as claimed in claim 38, wherein at least part of the securing means comprises at least one dimension corresponding to a dimension of known well-plate format and wherein the upper part comprises at least one dimension of a known well plate format.

42. The apparatus of claim 27, wherein the at least one outlet comprises a plurality of outlets and wherein at least one of a), b):
a) the plurality of outlets comprise at least one pair of outlets arranged at opposing sides of the collection reservoir;
b) at least one, optionally all, of the plurality of outlets are provided at and/or intersecting with a side wall of the collection reservoir.

43. The apparatus of claim 27, wherein the at least one outlet is provided in an annular region of the base portion, wherein the annular region is at least one of a), b), c):
a) beyond half of the radius of the base portion;
b) beyond three quarters of the radius to the edge of the base portion; c) substantially at the edge of the base portion.

44. A microfluidic device comprising:
a collection reservoir for collecting fluid, wherein the collection reservoir is at least partially closed at a first end by a base portion;
a fluid channel and an inlet coupling the fluid channel to the collection reservoir;
wherein the collection reservoir further comprises an at least one outlet for draining fluid from the collection reservoir, wherein at least part of the at least one outlet is provided on or adjacent to the base portion at the first end of the collection reservoir.

45. A material layer for use with at least one microfluidic device as claimed in claim 44, wherein the material layer comprises a plurality of absorbing and/or non-absorbing portions distributed to be interfaced with a corresponding plurality of outlets of one or more microfluidic devices.

46. A method of use of a microfluidic device, the microfluidic device comprising a collection reservoir for collecting fluid, wherein the collection reservoir is at least partially closed at a first end and comprises at least one outlet at or adjacent to the at least partially closed first end and a fluid channel and an inlet coupling the fluid channel to the collection reservoir; wherein the method comprises at least one of a), b):
a) providing the microfluidic device on a first material layer, wherein the first material layer comprises a non-absorbing portion such that at least one outlet of the microfluidic device is interfaced with the non-absorbing portion thereby to prevent or at least reduce flow from the microfluidic device;
b) providing the microfluidic device on a second material layer, wherein the second separate material layer comprises an absorbing portion such that the at least one outlet at the base of the microfluidic device is interfaced with the non-absorbing portion thereby to permit or at least increase fluid flow from the microfluidic device.
